(19) United States
(12) Patent Application Publication
(10) Pub. No.: US 2008/0138299 A1
(43) Pub. Date: Jun. 12, 2008

(54) Oral Pharmaceuticals or Oral Hygiene Products Comprising Licorice Flavonoid Extract

(75) Inventor: Yao Shi, Zhongshan (CN)

Correspondence Address: BAKER & MCKENZIE LLP, Pennzoil Place, South Tower, 711 Louisiana, Suite 3400, HOUSTON, TX, 77002-2716, US

(73) Assignee: Hawley & Hazel (BVI) Company Limited, Road Town (VG)

(21) Appl. No.: 11/950,386

(22) Filed: Dec. 4, 2007

(30) Foreign Application Priority Data
Dec. 4, 2006 (CN) 200610168413.6

(51) Int. Cl. A61K 36/484; A61K 8/97; A61Q 11/00

(52) U.S. Cl. 424/58; 424/757

(57) ABSTRACT

The present invention relates to the use of a compound Licochalone A (5-(1,1-Dimethylallyl)-4,4′-dihydroxy-2-methoxychalcone)) as well as Glycyrrhiza inflate Betal flavonoid extract, Glycyrrhiza glabra L. flavonoid extract, Glycyrrhiza kansuensis (Chang et Peng) flavonoid extract, a flavonoid extract of a mixture of two to three of the above licorices, or a mixture of two or three of the above licorice flavonoid extracts, which contains Licochalone A, in inhibiting primary oral pathogenic microorganisms and manufacturing oral care products for countering breath odor, preventing dental caries and reducing dental plaques, reducing gingivitis, tooth desensitizing, anti-calculus, and/or tooth whitening, and relates to oral pharmaceuticals and oral care products thereof.

### PRIOR RELATED APPLICATIONS

This application claims priority to Chinese Patent Application No. 200610168413.6 filed on Dec. 4, 2006.

### FIELD OF THE INVENTION

The present invention pertains to the field of pharmaceutical techniques, and relates to the inhibitive effect of Licochalone A (5-(1,1-Dimethylallyl)-4,4′-dihydroxy-2-methoxychalcone)) on primary oral pathogenic microorganisms and the application thereof in manufacturing oral pharmaceuticals or oral care products for countering breath odor, preventing dental caries, countering breath odor, reducing dental plaques, reducing gingivitis, tooth desensitizing, anti-calculus, and/or tooth whitening.

### BACKGROUND

The common human oral diseases include caries and periodontal disease. Caries is a chronic bacterial disease occurring in dental hard tissues, manifested as a color, shape, qualitative changes of dental hard tissues. Clinical symptoms of periodontal disease are gum bleeding, purulence, tooth mobility, absorption of alveolar bone and formation of periodontal pocket. The periodontal disease is also one of the primary causes for the tooth loss of adults. It's considered now that the pathogeny of both caries and periodontal disease is endogenous infection diseases resulted from a case that the normal oral floras lose balance, namely the initial factor being plaque microorganism, and being resulted from specific pathogenic microorganisms. The bacteria causing caries include Streptococcus mutans (S.m), Actinomyces nacslundi (A.n) and the like. The primary putative pathogenic microorganisms of periodontal disease include Porphyromonas gingivalis (P.g), Actinobacillus actinomycetemicomtans (A.a). The pharmaceuticals for treating caries and periodontal disease are very limited at present. On one hand, chemical pharmaceuticals are used to eliminate or inhibit the plaque bacteria, but the long-term use of broad-spectrum antibacterials will bring drug-resistant strains and other side effects. For example, the chemical plaque controlling agent Chlorhexidine used both at home and abroad in recent years is a diguanido-hexane having obvious bacterioslasis, but the long-term use of gargle containing 0.12-0.20% Chlorhexidine will cause stained tooth and formation of brown tongue coat at the back of tongue, although will not form any drug-resistant strains or cause any harm to human body. On the other hand, dialectic therapeutics of Traditional Chinese Medicines are used, such as gargling Chinese Medicine which is heat-clearing and detoxifying like honeysuckle, etc., as well as external-applying methods such as applying Bingpeng San (borneol and borax powder) or Asarum on the suffering part. There are many inconveniences in using these methods, and there is no conclusive evidence to prove their efficacy. However, if the gingivitis is not treated timely or properly, the inflammation symptom may spread to periodontal apex to cause the inflammation of periodontal apex tissues, and further affects the alveolar bone or the neighboring tissues. Based on the above reasons, it may be seen that it is meaningful to research and develop new medicines for curing common oral diseases such as caries and periodontal diseases, and to develop oral pharmaceuticals or oral care products for inhibiting primary oral pathogenic microorganisms, dental plaques, gingivitis, and for countering breath odor.

The researches on natural plants inhibiting oral pathogenic microorganisms have distinct geographical characteristics. In accordance with different geographical environments and country situations all over the globe, different countries and regions found some plants of the anti-caries effects, such as Anogeissuswall and Vernonia esculenta Hemsl, gamboge of Africa and the Middle East; blood grass roots, cocoa fruit of Latin American countries; betel nut of Southeast Asia; Eucalyptus leaves of Australian. China and Japan are more concentrated on the study of Chinese herbal medicines.

Licorice, which is the roots and rhizomes of fabaceous plant, is a traditional Chinese Medicine material and is widely used. It is mainly distributed in Xinjiang, Gansu and Inner Mongolia in China. The licorices produced in China containing Licochalone A mainly include Glycyrrhiza inflate Betal, Glycyrrhiza glabra L. and Glycyrrhiza kansuensis. The roots and rhizomes of licorice mainly contains triterpenes ingredients, dominated by glycyrrhizic acid, namely glycyrrhizin. Flavonoid compounds are also contained. So far, there is no report in prior art for using the compound licorice Chalcone A (5-(1,1-Dimethylallyl)-4,4′-dihydroxy-2-methoxychalcone) with a chemical formula (1) as an effective ingredient in the treatment of oral disease, nor report of the Use of the compound in preparing breath odor countering and anti-caries oral pharmaceuticals or oral care products, and in preparing anti-gingivitis, anti-plaque, tooth desensitizing, anti-calculus, and/or tooth whitening oral pharmaceuticals or oral care products.

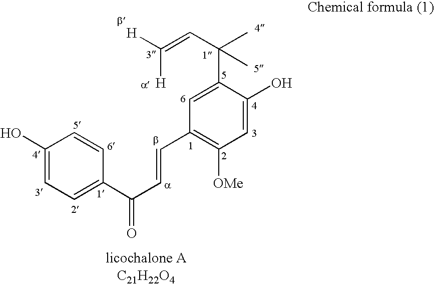

### SUMMARY OF THE INVENTION

The object of the present invention is to provide the inhibitive effect on oral pathogenic microorganisms of a compound Licochalone A (5-(1,1-Dimethylallyl)-4,4′-dihydroxy-2-methoxychalcone) and the Use of the compound Licochalone A as an effective ingredient in preparing anti-caries, breath odor countering oral pharmaceuticals or oral care products, and in preparing anti-gingivitis, anti-plaque, tooth desensitizing, anti-calculus, and/or tooth whitening oral pharmaceuticals or oral care products.

In order to achieve the above object, the present invention provides the following technical solution: the inhibitive effect of Licochalone A on the oral pathogenic microorganisms and the Use of Licochalone A as an effective ingredient in preparing anti-caries, breath odor countering oral pharmaceuticals or oral care products, and in preparing anti-gingivitis, anti-plaque, tooth desensitizing, anti-calculus, and/or tooth whitening oral pharmaceuticals or oral care products.

When the compound of the present invention is used as a pharmaceutical, it may be used directly with certain inhibition effect on oral pathogenic microorganisms, and may also be applied as the effective ingredient in oral pharmaceuticals or oral care products, such as tooth paste, mouthwash, paste, rapidly disintegrating tablets, pellicles and chewing gums and the like.

The present invention also researches on the Use of total flavonoid extract from its primary plant origin licorice in oral pharmaceuticals or oral care products, and optimizes the extraction process, finding that the total licorice flavonoid extract also have certain inhibition effect on oral pathogenic microorganisms, and may be applied in oral pharmaceuticals or oral care products as an natural anti-bacteria agent, which may effect to inhibit and destroy primary oral pathogenic microorganisms and to inhibit breath odor, anti-caries, counter plaques, densensitivate tooth, anti-calculus and/or whiten tooth.

The present invention provides the following technical solution: the inhibitive effect on primary oral pathogenic microorganisms and the Use of Zhangguo Licorice (Glycyrrhiza inflate Betal) flavonoid extract, Guangguo Licorice (Glycyrrhiza glabra L.) flavonoid extract, Huang Licorice (Glycyrrhiza kansuensis) flavonoid extract, a flavonoid extract of a mixture of two to three of the above licorices, or a mixture of two to three of the above licorice flavonoid extracts, which contain Licochalone A, in preparing oral pharmaceuticals or oral care products for breath odor countering, anti-caries and plaques reducing, gingivitis reducing, tooth desensitizing, anti-calculus, and/or tooth whitening and related oral pharmaceuticals or oral care products. The licorice flavonoid extract herein refers to the extract extracted through a series of methods, mainly flavonoid compound (more than 40%) and preferentially containing no or a small quantity (no more than 30%) of triterpenoids.

### BRIEF DESCRIPTION OF THE DRAWINGS

This invention can be better understood in connection with the accompanying drawings, which non-exhaustively illustrate without limitation certain embodiments of this invention and include:

FIG. 1 illustrates a process to extract flavonoid from Zhangguo Licorice powder.

### DETAILED DESCRIPTIONS OF THIS INVENTION

In order to understand the essence of the present invention, the following will use the compound Licochalone A with the chemical formula (1) and the total flavonoid extract of Glycyrrhiza inflate Betal, Glycyrrhiza glabra L. or Glycyrrhiza kansuensis, the flavonoid extract of the mixture of two to three of the above licorices, or the mixture of two to three of the above licorice flavonoid extracts and their inhibitive effects on primary oral pathogenic microorganisms and anti-inflammatory results after applied in oral pharmaceuticals or oral care products to illustrate the essence of the present invention, but the present invention is not so limited.

1. The Extraction of Total Flavonoid Extracts of Zhangguo Licorice (Glycyrrhiza inflate Betal)

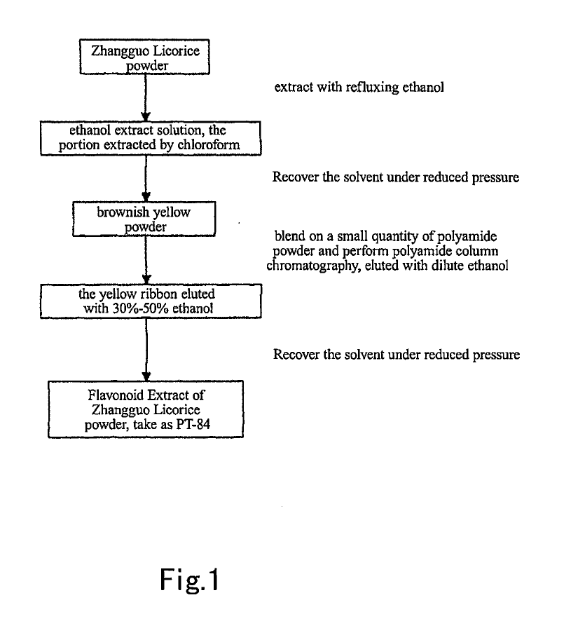

FIG. 1 illustrates a process to extract flavonoid from Zhangguo Licorice powder. The rhizomes of Zhangguo Licorice (whose scientific name is identified as Glycyrrhiza inflate Betal) collected from Xinjiang is dried in the shade at room temperature and pulverized moderately, extracted three times by heating and reflux using 95% ethanol, with 3 h each time, and the alcohol extract solutions are combined. Recover under reduced pressure until there is no obvious smell of alcohol. After dissolving with appropriate amount of water, extract using in the order of petroleum ether and chloroform. Extract is obtained from the resultant chloroform part after partially recovering solvent, wherein this part mainly contains triterpenoids such as glycyrrhizic acid, and also flavanoids. The extractum is subjected to polyamide column chromatography, eluted with 30%-50% ethanol, and the enriched licorice flavonoid extract is obtained.

### 2. The Extraction and Identification of Licochalone A

The total flavonoid extract PT-84 was first obtained using the method of Example 1. PT84 (100 g) is weighted, dissolved in methanol, and silica gel for column chromatography (100-200 mesh) 100 g is weighted to blend sample. Evaporate the solvent, finely ground, pass a sieve with mesh size of 80 and stay until use. Weight silica gel for column chromatography (100-200 mesh) 1.5 kg, and fill the column with chloroform using wet method. The sample staying for use is applied to the column and is separated by using silica gel column chromatography. The eluents are in a sequence of: chloroform-ethyl acetate (100:4), chloroform-ethyl acetate (100:5), chloroform-ethyl acetate (100:6), chloroform-ethyl acetate (100:6.5), chloroform-ethyl acetate (100:7), ethyl acetate, and methanol. After repeated silica gel column chromatography, gel column chromatography, and recrystallization, a yellow needle crystalline compound Licochalone A is obtained. With TLC silica gel F₂₅₄ sheet detection, the Rf value is the same as the reference Licochalone A.

### The Structural Data of Licochalone A

Structure: As shown in chemical formula (1)

### Molecular Formula: C₂₁H₂₂O₄

### Molecular Weight: 338

Properties: small yellow needle crystal (ethanol)

Mₚ: 93-95° C.

UV(MeOH) λₘₐₓ: 262, 308, 378, 250, 274, 350 (NaOMe) nm

IR(KBr)v 3437, 2965, 2925, 1637, 1603, 1560, 1509, 1446, 1396, 1344, 1288, 1215, 1166, 1077, 1034, 906, 835 cm⁻¹

¹H-NMR δ 8.00 (1H, d, J=15.6 Hz, β-H), 7.98 (2H, d, J=8.5 Hz, H-2′, 6′), 7.58 (1H, d, J=15.6 Hz, H-α), 7.42 (1H, s, H-6), 6.96 (2H, d, J=8.5 Hz, H-3′, 5′), 6.41 (1H, dd, J=10.4, 17.8 Hz, H-3), 6.15 (1H, s, H-2″), 5.25 (1H, d, J=17.8 Hz, H-3″(α′)), 5.21 (1H, d, J=10.4 Hz, H-3″(β′)), 3.75 (3H, s, —OCH₃), 1.40 (6H, s, H-4″,5″)

¹³C-NMR (CDCl₃) δ191.0 (C═O), 161.4 (C-4′), 159.4 (C-4), 158.6 (C-2), 147.6 (C-2″), 141.6 (C-β), 131.2 (C-2′, 6′), 130.4 (C-1′), 128.9 (C-6), 125.1 (C-5), 119.7 (C-α), 116.0 (C-1), 115.6 (C-3,5′), 113.2 (C-3′), 100.9 (C-3), 55.6 (—OCH₃), 39.8 (C-1″), 27.1 (C-4″, 5″)

In accordance with the compound structure formula disclosed in the present invention, those skilled in the art may synthesize said compound easily by chemical methods without creative work, to substitute the extract or purified substance used in the present invention. Therefore, those skilled in the art should understand that, in addition to extraction or purification from natural licorices listed in the examples of the present invention, the present invention may also be achieved through chemical synthesis methods. Other obvious modification of the compound disclosed in the present invention will also be included within the protection scope of the present invention.

3. The Inhibition Experiment of Licochalone A and Total Flavonoid Extracts of Zhangguo Licorice (Glycyrrhiza inflate Betal) on Primary Oral Pathogenic Microorganisms
3.1 The Cultivation of the Bacteria Species: Streptococcus mutans (ATCC 25175), Actinomyces naeslundii (ATCC 12104), Actinobacillus actinomycetemicomtans (ATCC 43717), Porphyromonas gingivalis (ATCC 33277), Listed as Following:

TABLE 1
Related Oral Pathogenic microorganisms

| | | Gram |
|---|---|---|
| Strains | ATCC number | strain |
| Actinomyces naeslundii | 12104 | G(+) |
| (A. n) | | |
| Actinobacillus | 43717 | G(−) |
| actinomycetemcomitans | | |
| (A. a) | | |
| Streptococcus mutans | 25175 | G(+) |
| (S. m) | | |
| Porphyromonas gingivalis | 33277 | G(−) |
| (P. g) | | |

A single colony is picked from the Trypticase Digested Soybean peptone agar blood plate (TSA5B) ordinarily stored bacteria species to Trypticase Digested Soybean peptone broth (TSB), cultivated in microaerobic environment (only P.g needs anaerobic cultivation) with 95% air, 5% CO₂ at 37.0° C.±1.0° C., wherein S.m is cultivated for 18-24 h, and the remaining three are cultivated for 40-48 h. Then sterile TSB is used to adjust the turbidity of the bacteria suspension to 0.5^{#}McFarland standard, equivalent to 1.0×10⁸ CFU/mL.

### 3.2 Broth Dilution Method (Broth Dilution)

After performing a series of 2-fold dilutions to the anti-bacteria pharmaceuticals, rationally inoculate with tested bacteria, and observe after incubated at 37° C. for 18-24 h, wherein the lowest pharmaceutical concentration that inhibits the growth visible to the naked eye of the tested bacteria is the minimum inhibitive concentration (MIC) of the tested pharmaceuticals to the tested bacteria. The operating steps are:

a. Preparation of the stock solution of antibacterial pharmaceutical: Prepare 1% stock solution of various antibacterial pharmaceuticals dope with 100% pure ethanol as solvent. The prepared stock solution is filtered to eliminate bacteria, and is distributed into small portions to be used.
b. Range of measured concentration: the present experiment chooses 250 ppm as the ceiling measured concentration of the anti-bacteria pharmaceutical.
c. Measuring method: micro-dilution method.

First add 100 μL TSB to each hole of the 96-well plate, and add 100 μL sterile anti-bacteria pharmaceutical solution which is diluted 10-folds (1000 mg/l) to each hole of the first column, then blow on each hole of the first column repeatedly for 7-8 times using multi-channel micropipet, transfer 100 μL sample to the second column after sufficiently mixing the pharmaceutical with TSB, and after similarly blowing for 7-8 times, transfer 100 μL sample to the third column, and analogously to the last column, thus the concentration of pharmaceutical is 2-fold gradiently diluted, from 500 mg/L of the first column to 0.24 mg/L of the last column (the 12th column).

The bacteria to be tested and standard bacteria are prepared as above. Dilute the bacteria solution with TSB to a bacteria content of about 10⁶ CFU/mL. Then inoculate 100 μL for each hole. Thus the final diluted concentration of the anti-bacteria pharmaceutical in each line are 250, 125, 62.5 . . . 0.12 mg/L, and the final inoculation quantity is about 5×10⁷ CFU/mL or 5×10⁶ bacteria each hole; the 96-well plate is placed on a microshaker and is shook for 1 min to mix the solution in each hole. The microwell plate is covered and is sealed with adhesive paper to reduce the evaporation during the incubation process and is placed in a humid box, cultivated in microaerobic environment with 95% air, 5% CO₂ (or in anaerobic environment with 90% N₂, 5% H₂, 5% CO₂) at 37.0° C.±1.0° C. for 18-24 h. The 96-well plate is placed under an enzyme-labeling instrument, and by comparing the growing characteristics of the tested bacteria with the standard bacteria, the lowest anti-bacteria pharmaceutical concentration contained in the hole without bacteria is taken as MIC.

Result: Both Licochalone A and total flavonoid extract of Zhangguo Licorice have very strong anti-bacteria ability. Refer to Table 2 for detailed result.

TABLE 2
(The smaller the value, the stronger the anti-bacteria ability)

| Extract/Compound | A. a | A. n | S. m | P. g |
|---|---|---|---|---|
| Total flavonoid extract of | >250 | 15.6 | 31.25 | 1.9 |
| Zhangguo Licorice | | | | |
| (PT-84, total flavones 60%) | | | | |
| Licochalone A | >250 | 7.8 | 7.8 | 1.9 |
| Triclosan | 1.9 | 3.9 | 3.9 | 7.8 |
| (Positive Control) | | | | |

4. Anti-Inflammatory Experiment of Licochalone A and Total Flavonoid Extract of Zhangguo Licorice (Glycyrrhiza inflate Betal)

### 4.1 The Experiment Using KB Cell (the Epidermic Cancer Cell in Human Oral Cavity):

During the experiment, the KB cell inoculated in the cultivating plate is treated with or without (control) Licochalone A. After the treatment, the supernatant of the cultivated liquid is collected and stored in −80 C refrigerator. The PGE₂ in the supernatant is detected by Enzyme-linked immunsorbent assay, and Luminex multifunctional liquid-phase chip analysis system is used for the detection of GM-CSF, TNF-α, IL-1β and IL-6 (the result is shown in Table 3).

### 4.2 Detection of PGE₂ by Enzyme-Linked Immunsorbent Assay: Performed as Standard Enzyme-Linked Immunsorbent Assay.

Result (the smaller the value, the stronger the anti-inflammatory activity): The median inhibitive concentration of Licochalone A to the growth of oral KB cell is 0.02 ppm, equivalent to the positive control Triclosan. The median inhibitive concentration of PT-84 to the growth of oral KB cell is 0.12 ppm. The result shows that both Licochalone A and total flavonoid extract of Zhangguo Licorice PT-84 have significant anti-inflammatory activity.

### 4.3 Luminex Multifunctional Liquid-Phase Chip Analysis of GM-CSF, TNF-α, IL-1 and IL-6:

The 96-well plate is enclosed for 30 min. The fluorescent microspheres are added into the 96-well plate after dilution. Add the standard sample and the sample to be measured, stay overnight at 4° C. In the next day, the supernatant is discarded, and 50 μl GM-CSF, TNF-α, IL-1β and IL-6 antibodies are added to each hole. After rinsing 4 times, the 96-well plate is placed in a shaking table under ambient temperature to shade from light for 1 h, rinse 4 times, and add PE labeled with streptavidin and shade from light for 15 min at room temperature. Rinse 4 times, the microspheres are suspended in the rinsing buffer, and the 96-well plate is immediately placed on Luminex (Luminex Corporation, Austin, Tx.) to analyze.

Result: Both Licochalone A and PT-84 have very strong anti-inflammatory ability. The result is shown in Table 3.

TABLE 3
(the higher the value, the stronger the anti-inflammatory ability)

| | | | | TNF-α |
|---|---|---|---|---|
| | | | | (Tumor |
| | | IL-1β | IL-6 | Necrosis |
| Extract/Compound | GM-CSF | (interleukin) | (interleukin) | Factor) |
| Total flavonoid | 20% | 23% | 8% | 68% |
| extract of Zhangguo | | | | |
| Licorice(PT-84) | | | | |
| (total flavones 60%) | | | | |
| Licochalone A | 8% | 19% | 50% | 95% |
| Triclosan | 5% | 7% | 31% | 81% |
| (Positive Control) | | | | |

It can be proved from the above experiment results that: the Licochalone A and total flavonoid extract of Zhangguo Licorice (Glycyrrhiza inflate Betal) have excellent inhibitive effect on oral pathogenic microorganisms (Actinomyces naeslundii, Actinobacillus actinomycetemcomitans, Streptococcus mutans, Porphyromonas gingivalis), and have significant anti-inflammatory activity to oral KB cells, and also have evident inhibitive effect on inflammatory factors.

### 5. Use of Licochalone A and Total Flavonoid Extract of Zhangguo Licorice (Glycyrrhiza Inflate Betal) in Oral Hygiene Products

The above resultant flavonoid extract of Zhangguo Licorice (Glycyrrhiza inflate Betal), or Licochalone A obtained by further purification and separation may be mixed in effective amount with other ingredients, such as humectants, abrasives, surfactants, pharmacy acceptable carriers and/or additives, to make oral pharmaceuticals or oral hygiene products, the resultant oral pharmaceuticals or oral hygiene products include, but not limited to, toothpaste, mouthwash, gum and oral paste, etc. The effective amount referred herein is a dose that is sufficient to produce a positive effect.

The composition of the formulation involved in the present invention may further contain cationic surfactants and/or non-ionic surfactants. Cationic surfactants include, but are not limited to, cetyltrimethylammonium chloride, di(isobutyl-phenoxy-ethoxy-ethyl)-di(xylene-benzyl) ammonium bromide. Non-ionic surfactants include Poloxamer, polyethoxy ether, and ethoxy fatty acid and the like. Poloxamer is the block copolymer of polyoxyethylene and polyoxypropylene, having industrial applications, for example, produced by BASF under the trade name “Pluronic”. Polyethoxy ether includes polyoxyethylene sorbitol fatty acid ester (a typical monoester of polyethoxy alcohol), which is produced by ICL company with the trade name Tween. Other non-ionic surfactants include polyoxyethylene alkylphenol, polyoxyethylene alcohol, fatty acids, polyoxyethylene ester, polyoxyethylene alkyl amines glyceride, polyglyceride, tetritol ester, pentitol ester, hexitol ester, dehydrate tritol ester and polyoxyalkylamine polyalkyl ester. Typically, the mass content of surfactant in the formulation is 0.001%-3.0%.

The formulation may contain divalent metal ions such as zinc, copper, selenium, calcium or magnesium. They will be in the form of soluble inorganic salts such as zinc chloride or the form of organic/inorganic compounds. The amount of divalent metal ions added into the mixture will be 0.001%-3.0%.

The formulation may contain oligosaccharides, which will be in the soluble state, and may convert into soluble salt. If added, in amount of at least 0.01%.

The present invention includes various commonly used oral pharmaceuticals or oral hygiene products, such as: mouthwash, oral spray, toothpaste, gels, oral mucosa plasters, oral pellicle, rapidly disintegrating tablet and pellicle, chewing gum and the like for mammals, especially human and animals, to inhibit plaque and prevent dental caries and periodontal diseases.

Other additives may be used due to different methods: mouthwash may contain acidic substances; mouthwash may also contain desensitizing substance such as potassium nitrate; toothpaste may contain abrasives such as sodium carbonate, calcium phosphate, alumina, silica; solubilizer such as PEG, glycerol, ethanol, other taste masking agents such as xylitol, thickener such as carrageenan, and surfactant such as SLS may all be used.

Waxes such as beeswax may be used both in toothpaste and oral mucosa plaster; thickener and pellicle-forming agents such as Carrageenan, CMC, HPMC, xanthan gum and the like may also be added.

Essences may be used, such as peppermint, spearmint, eucalyptus leaf oil, menthol, carvone, wintergreen, cloves, cinnamon, lemon, grapefruit, orange and some essences decomposed and digested from yeast and protein.

In tablets and pellicles, inert excipients may be used for the formation of the products.

For other additives well-known to those skilled in the art that may be used for oral hygiene products of the present invention, one can refer to published PCT international application WO01/82922A1. To save space, the full text of WO01/82922A1 is incorporated into the present application by reference, as if the disclosure of which is listed herein word by word.

In tooth paste formulation, the flavonoid extract of Zhangguo Licorice (Glycyrrhiza inflate Betal) may be mixed with other ingredients such as humectants (such as sorbitol), abrasives (such as silica), surfactants (such as sodium dodecyl sulfate) and the like in a amount of 0.0005-20% (mass percentage). Preferably, the content of flavonoid extract of Zhangguo Licorice (Glycyrrhiza inflate Betal) is 0.001-10% (mass percentage). More preferably, the content of flavonoid extract of Zhangguo Licorice (Glycyrrhiza inflate Betal) is 0.025-5% (mass percentage). Especially preferably, the content of flavonoid extract of Zhangguo Licorice (Glycyrrhiza inflate Betal) is 0.5% (mass percentage). The content of Licochalone A in tooth paste may be 0.0005-20% (mass percentage), preferably 0.001-10%, more preferably 0.025-5% (mass percentage), and especially preferably 0.3%-0.5% (mass percentage).

In mouthwash formulation, the flavonoid extract of Zhangguo Licorice (Glycyrrhiza inflate Betal) may be mixed with other major ingredients such as alcohol, Pluronic, and/or other mouthwash additives in a amount of 0.0005-20% (mass percentage). Preferably, the content of flavonoid extract of Zhangguo Licorice (Glycyrrhiza inflate Betal) is 0.001-5% (mass percentage). More preferably, the content of flavonoid extract of Zhangguo Licorice (Glycyrrhiza inflate Betal) is 0.025-0.5% (mass percentage). Especially preferably, the content of flavonoid extract of Zhangguo Licorice (Glycyrrhiza inflate Betal) is 0.05% (mass percentage). The content of Licorice Chalcone A in mouthwash formula may be 0.0005-20% (mass percentage), preferably 0.001-5% (mass percentage), more preferably 0.025-0.5% (mass percentage), especially preferably 0.05% (mass percentage).

In oral paste formulation, the flavonoid extract of Zhangguo Licorice (Glycyrrhiza inflate Betal) may be mixed with other major ingredients such as polyethylene glycol and/or other oral paste additives in a amount of 0.0005-20% (mass percentage). Preferably is 0.001-10% (mass percentage). More preferably, the content of flavonoid extract of Zhangguo Licorice (Glycyrrhiza inflate Betal) is 0.01-5% (mass percentage). Especially preferably, the content of flavonoid extract of Zhangguo Licorice (Glycyrrhiza inflate Betal) is 0.025% (mass percentage). The content of Licorice Chalcone A in mouth pastes formulation may be 0.0005-20% (mass percentage), preferably 0.001-10% (mass percentage), more preferably 0.01-5% (mass percentage), especially preferably 0.025% (mass percentage).

The following will be combined the examples to further illustrate the essential content of the present invention, however, the content of the present invention is not so limited.

### EXAMPLE 1

### Use of the Flavonoid Extract of Zhangguo Licorice (Glycyrrhiza inflate Betal) in Tooth Paste Formulation

Tooth paste formulation 1 of flavonoid extract of Zhangguo Licorice (Glycyrrhiza inflate Betal):

| | Content |
|---|---|
| Components | (Mass percentage, %) |
| Flavonoid extract of Zhangguo Licorice | 0.0005-20 |
| PT-84 | |
| abrasive | 10-30 |
| humectant | 50-70 |
| Water, flavors, flavorings | 0-proper amount |

Tooth paste formulation 2 of flavonoid extract of Zhangguo Licorice (Glycyrrhiza inflate Betal):

| | Content |
|---|---|
| Components | (Mass percentage, %) |
| Flavonoid extract of Zhangguo Licorice | 5 |
| PT-84 | |
| Silica dioxide | 30 |
| Glycerine | 5 |
| Sorbitol | 50 |
| Water, flavors, flavorings | proper amount |

Tooth paste formulation 3 of flavonoid extract of Zhangguo Licorice (Glycyrrhiza inflate Betal):

| | Content |
|---|---|
| Components | (Mass percentage, %) |
| Flavonoid extract of Zhangguo Licorice | 0.001 |
| PT-84 | |
| Silica dioxide | 10 |
| Sorbitol | 70 |
| Water, flavors, flavorings | proper amount |

Tooth paste formulation 4 of flavonoid extract of Zhangguo Licorice (Glycyrrhiza inflate Betal):

| | Content |
|---|---|
| Components | (Mass percentage, %) |
| Flavonoid extract of Zhangguo Licorice | 10 |
| PT-84 | |
| abrasive | 25 |
| humectant | 65 |

Tooth paste formulation 5 of flavonoid extract of Zhangguo Licorice (Glycyrrhiza inflate Betal):

| | Content |
|---|---|
| Components | (Mass percentage, %) |
| Flavonoid extract of Zhangguo Licorice | 0.025 |
| PT-84 | |
| Silica dioxide | 20 |
| Glycerine | 5 |
| Sorbitol | 60 |
| Sodium fluoride | 0.221 |
| Saccharin sodium | 0.3 |
| Water | 7.954 |
| Polyethylene glycol | 3 |
| Sodium dodecyl sulfate | 2 |
| Essence | 1.5 |

Tooth paste formulation 6 of flavonoid extract of Zhangguo Licorice (Glycyrrhiza inflate Betal):

| | Content |
|---|---|
| Components | (Mass percentage, %) |
| Flavonoid extract of Zhangguo Licorice | 0.0005 |
| PT-84 | |
| Silica dioxide | 10 |
| Sorbitol | 70 |
| Water, flavors, flavorings | proper amount |

Tooth paste formulation 7 of flavonoid extract of Zhangguo Licorice (Glycyrrhiza inflate Betal):

| | Content |
|---|---|
| Components | (Mass percentage, %) |
| Flavonoid extract of Zhangguo Licorice | 20 |
| PT-84 | |
| abrasive | 25 |
| humectant | 55 |

Tooth paste formulation 8 of flavonoid extract of Zhangguo Licorice (Glycyrrhiza inflate Betal):

| | Content |
|---|---|
| Components | (Mass percentage, %) |
| Flavonoid extract of Zhangguo Licorice | 0.5 |
| PT-84 | |
| Silica dioxide | 20 |
| Glycerine | 5 |
| Sorbitol | 60 |
| Sodium fluoride | 0.221 |
| Saccharin sodium | 0.3 |
| Water | 7.479 |
| Polyethylene glycol | 3 |
| Sodium dodecyl sulfate | 2 |
| Essence | 1.5 |

### EXAMPLE 2

### Use of Licochalone A in Tooth Paste Formulation

Tooth paste formulation 1 of Licochalone A:

| | Content |
|---|---|
| Components | (Mass percentage, %) |
| Licochalone A | 0.0005-20 |
| abrasive | 10-30 |
| humectant | 50-69 |
| Water, flavors, flavorings | proper amount |

Tooth paste formulation 2 of Licochalone A

| | Content |
|---|---|
| Components | (Mass percentage, %) |
| Licochalone A | 5 |
| Silica dioxide | 30 |
| Glycerine | 5 |
| Sorbitol | 50 |
| Water, flavors, flavorings | proper amount |

Tooth paste formulation 3 of Licochalone A:

| | Content |
|---|---|
| Components | (Mass percentage, %) |
| Licochalone A | 0.001 |
| Silica dioxide | 10 |
| Sorbitol | 69 |
| Water, flavors, flavorings | proper amount |

Tooth paste formulation 4 of Licochalone A:

| | Content |
|---|---|
| Components | (Mass percentage, %) |
| Licochalone A | 10 |
| abrasive | 25 |
| humectant | 65 |

Tooth paste formulation 5 of Licochalone A:

| | Content |
|---|---|
| Components | (Mass percentage, %) |
| Licochalone A | 0.0005 |
| Silica dioxide | 10 |
| Sorbitol | 70 |
| Water, flavors, flavorings | proper amount |

Tooth paste formulation 6 of Licochalone A:

| | Content |
|---|---|
| Components | (Mass percentage, %) |
| Licochalone A | 20 |
| abrasive | 25 |
| humectant | 55 |

Tooth paste formulation 7 of Licochalone A:

| | Content |
|---|---|
| Components | (Mass percentage, %) |
| Licochalone A | 0.025 |
| Silica dioxide | 20 |
| Glycerine | 5 |
| Sorbitol | 60 |
| Sodium fluoride | 0.221 |
| Saccharin sodium | 0.3 |
| Water | 7.954 |
| Polyethylene glycol | 3 |
| Sodium dodecyl sulfate | 2 |
| Essence | 1.5 |

Tooth paste formulation 8 of Licochalone A

| | Content |
|---|---|
| Components | (Mass percentage, %) |
| Licochalone A | 0.3 |
| Silica dioxide | 20 |
| Glycerine | 5 |
| Sorbitol | 60 |
| Sodium fluoride | 0.221 |
| Saccharin sodium | 0.3 |
| Water | 7.679 |
| Polyethylene glycol | 3 |
| Sodium dodecyl sulfate | 2 |
| Essence | 1.5 |

Tooth paste formulation 9 of Licochalone A

| | Content |
|---|---|
| Components | (Mass percentage, %) |
| Licochalone A | 0.5 |
| Silica dioxide | 20 |
| Glycerine | 5 |
| Sorbitol | 60 |
| Sodium fluoride | 0.221 |
| Saccharin sodium | 0.3 |
| Water | 7.479 |
| Polyethylene glycol | 3 |
| Sodium dodecyl sulfate | 2 |
| Essence | 1.5 |

### EXAMPLES 3

### Use of the Flavonoid Extract of Zhangguo Licorice (Glycyrrhiza inflate Betal) in Mouthwash Formulation

Mouthwash formulation 1 of flavonoid extract of Zhangguo Licorice (Glycyrrhiza inflate Betal):

| | Content |
|---|---|
| Components | (Mass percentage, %) |
| Flavonoid extract of Zhangguo Licorice | 0.0005-20 |
| PT-84 | |
| Water | 70-94 |
| Other additives | proper amount |

Mouthwash formulation 2 of flavonoid extract of Zhangguo Licorice (Glycyrrhiza inflate Betal):

| | Content |
|---|---|
| Components | (Mass percentage, %) |
| Flavonoid extract of Zhangguo Licorice | 5 |
| PT-84 | |
| Water | 80 |
| Pluronic | 3.00 |
| Other additives | proper amount |

Mouthwash formulation 3 of flavonoid extract of Zhangguo Licorice (Glycyrrhiza inflate Betal):

| | Content |
|---|---|
| Components | (Mass percentage, %) |
| Flavonoid extract of Zhangguo Licorice | 0.001 |
| PT-84 | |
| Water | 94 |
| Alcohol | 5.00 |
| Other additives | proper amount |

Mouthwash formulation 4 of flavonoid extract of Zhangguo Licorice (Glycyrrhiza inflate Betal):

| | Content |
|---|---|
| Components | (Mass percentage, %) |
| Flavonoid extract of Zhangguo Licorice | 0.5 |
| PT-84 | |
| Water | 90 |
| Pluronic | 3.00 |
| Alcohol | 3.00 |
| Other additives | proper amount |

Mouthwash formulation 5 of flavonoid extract of Zhangguo Licorice (Glycyrrhiza inflate Betal):

| | Content |
|---|---|
| Components | (Mass percentage, %) |
| Flavonoid extract of Zhangguo Licorice | 0.025 |
| PT-84 | |
| Water | 85 |
| Pluronic | 5.00 |
| Alcohol | 3.00 |
| Other additives | proper amount |

Mouthwash formulation 6 of flavonoid extract of Zhangguo Licorice (Glycyrrhiza inflate Betal):

| | Content |
|---|---|
| Components | (Mass percentage, %) |
| Flavonoid extract of Zhangguo Licorice | 0.05 |
| PT-84 | |
| Water | 91.7 |
| Pluronic | 3.00 |
| Alcohol | 5.00 |
| Essence | 0.25 |

Mouthwash formulation 7 of flavonoid extract of Zhangguo Licorice (Glycyrrhiza inflate Betal):

| | Content |
|---|---|
| Components | (Mass percentage, %) |
| Flavonoid extract of Zhangguo Licorice | 0.0005 |
| PT-84 | |
| Water | 94 |
| Alcohol | 5.00 |
| Other additives | proper amount |

Mouthwash formulation 8 of flavonoid extract of Zhangguo Licorice (Glycyrrhiza inflate Betal):

| | Content |
|---|---|
| Components | (Mass percentage, %) |
| Flavonoid extract of Zhangguo Licorice | 20 |
| PT-84 | |
| Water | 70 |
| Pluronic | 3.00 |
| Alcohol | 3.00 |
| Other additives | proper amount |

### EXAMPLE 4

### Use of the Licochalone A in Mouthwash Formulation

Mouthwash formulation 1 of Licochalone A:

| | Content |
|---|---|
| Components | (Mass percentage, %) |
| Licochalone A | 0.0005-20 |
| Water | 70-94 |
| Other additives | proper amount |

Mouthwash formulation 2 of Licochalone A:

| | Content |
|---|---|
| Components | (Mass percentage, %) |
| Licochalone A | 5 |
| Water | 80 |
| Pluronic | 3.00 |
| Other additives | proper amount |

Mouthwash formulation 3 of Licochalone A:

| | Content |
|---|---|
| Components | (Mass percentage, %) |
| Licochalone A | 0.001 |
| Water | 94 |
| Alcohol | 5.00 |
| Other additives | proper amount |

Mouthwash formulation 4 of Licochalone A:

| | Content |
|---|---|
| Components | (Mass percentage, %) |
| Licochalone A | 0.5 |
| Water | 90 |
| Pluronic | 3.00 |
| Alcohol | 3.00 |
| Other additives | proper amount |

Mouthwash formulation 5 of Licochalone A:

| | Content |
|---|---|
| Components | (Mass percentage, %) |
| Licochalone A | 0.025 |
| Water | 85 |
| Pluronic | 5.00 |
| Alcohol | 3.00 |
| Other additives | proper amount |

Mouthwash formulation 6 of Licochalone A:

| | Content |
|---|---|
| Components | (Mass percentage, %) |
| Licochalone A | 0.05 |
| Water | 91.7 |
| Pluronic | 3.00 |
| Alcohol | 5.00 |
| Essence | 0.25 |

Mouthwash formulation 7 Licochalone A:

| | Content |
|---|---|
| Components | (Mass percentage, %) |
| Licochalone A | 0.0005 |
| Water | 94 |
| Alcohol | 5.00 |
| Other additives | proper amount |

Mouthwash formulation 8 of Licochalone A:

| | Content |
|---|---|
| Components | (Mass percentage, %) |
| Licochalone A | 20 |
| Water | 70 |
| Pluronic | 3.00 |
| Alcohol | 3.00 |
| Other additives | proper amount |

### EXAMPLE 5

### Use of the Flavonoid Extract of Zhangguo Licorice (Glycyrrhiza inflate Betal) in Paste Formulation

Paste formulation 1 of flavonoid extract of Zhangguo Licorice (Glycyrrhiza inflate Betal):

| | Content |
|---|---|
| Components | (Mass percentage, %) |
| Flavonoid extract of Zhangguo Licorice | 10 |
| PT-84 | |
| Polyethylene glycol | 90 |

Paste formulation 2 of flavonoid extract of Zhangguo Licorice (Glycyrrhiza inflate Betal):

| | Content |
|---|---|
| Components | (Mass percentage, %) |
| Flavonoid extract of Zhangguo Licorice | 0.001 |
| PT-84 | |
| Polyethylene glycol | 80 |
| Other additives | proper amount |

Paste formulation 3 of flavonoid extract of Zhangguo Licorice (Glycyrrhiza inflate Betal):

| | Content |
|---|---|
| Components | (Mass percentage, %) |
| Flavonoid extract of Zhangguo Licorice | 0.01 |
| PT-84 | |
| Polyethylene glycol | 90 |
| Other additives | proper amount |

Paste formulation 4 of flavonoid extract of Zhangguo Licorice (Glycyrrhiza inflate Betal):

| | Content |
|---|---|
| Components | (Mass percentage, %) |
| Flavonoid extract of Zhangguo Licorice | 5 |
| PT-84 | |
| Polyethylene glycol | 95 |

Paste formulation 5 of flavonoid extract of Zhangguo Licorice (Glycyrrhiza inflate Betal):

| | Content |
|---|---|
| Components | (Mass percentage, %) |
| Flavonoid extract of Zhangguo Licorice | 0.025 |
| PT-84 | |
| Polyethylene glycol | 99.975 |

Paste formulation 6 of flavonoid extract of Zhangguo Licorice (Glycyrrhiza inflate Betal):

| | Content |
|---|---|
| Components | (Mass percentage, %) |
| Flavonoid extract of Zhangguo Licorice | 20 |
| PT-84 | |
| Polyethylene glycol | 80 |

Paste formulation 7 of flavonoid extract of Zhangguo Licorice (Glycyrrhiza inflate Betal):

| | Content |
|---|---|
| Components | (Mass percentage, %) |
| Flavonoid extract of Zhangguo Licorice | 0.0005 |
| PT-84 | |
| Polyethylene glycol | 99 |

### EXAMPLE 6

### Use of Licochalone A in Paste Formulation

Paste formulation 1 of flavonoid extract of Licochalone A:

| | Content |
|---|---|
| Components | (Mass percentage, %) |
| Licochalone A | 10 |
| Polyethylene glycol | 90 |

Paste formulation 2 of flavonoid extract of Licochalone A:

| | Content |
|---|---|
| Components | (Mass percentage, %) |
| Licochalone A | 0.001 |
| Polyethylene glycol | 80 |
| Other additives | proper amount |

Paste formulation 3 of flavonoid extract of Licochalone A:

| | Content |
|---|---|
| Components | (Mass percentage, %) |
| Licochalone A | 0.01 |
| Polyethylene glycol | 90 |
| Other additives | proper amount |

Paste formulation 4 of flavonoid extract of Licochalone A:

| | Content |
|---|---|
| Components | (Mass percentage, %) |
| Licochalone A | 5 |
| Polyethylene glycol | 95 |

Paste formulation 5 of flavonoid extract of Licochalone A:

| | Content |
|---|---|
| Components | (Mass percentage, %) |
| Licochalone A | 0.025 |
| Polyethylene glycol | 99.975 |

Paste formulation 6 of flavonoid extract of Licochalone A:

| | Content |
|---|---|
| Components | (Mass percentage, %) |
| Licochalone A | 20 |
| Polyethylene glycol | 80 |

Paste formulation 7 of flavonoid extract of Licochalone A:

| | Content |
|---|---|
| Components | (Mass percentage, %) |
| Licochalone A | 0.0005 |
| Polyethylene glycol | 99 |

The above detailed description of the present invention sets forth a lot of specified details, the object thereof is to give clear and integrated explanation of the invention to facilitate the public to read and understand. However, what will be clear to one skilled in the art is that in some cases, the present invention may also be practiced without these specified details, or by making some non-essential modification or substitution to these specified details. These variations should be all considered to fall into the spirit and scope of the present invention. Moreover, the extracting or purifying solution shown in the form of block diagram and flow chart is only illustrative embodiment, only to illustrate the invention, rather than to limit the invention. The scope of the invention should be determined by the plain text of the appended claims, rather than limited to the particular examples provided in the description.

What is claimed is:

1. An oral pharmaceutical or oral care product, characterized in comprising a licorice flavonoid extract, wherein the licorice is selected from Glycyrrhiza inflate Betal, Glycyrrhiza glabra L. and Glycyrrhiza kansuensis, and a mixture thereof.

2. The oral pharmaceutical or oral care product of claim 1, characterized in that the licorice flavonoid extract is in an amount of 0.0005 wt %-20 wt %.

3. The oral pharmaceutical or oral care product of claim 1, characterized in that the licorice flavonoid extract is in an amount of 0.001 wt %-10 wt %.

4. The oral pharmaceutical or oral care product of claim 1, characterized in that the licorice flavonoid extract is in an amount of 0.025 wt %-5 wt %.

5. The oral pharmaceutical or oral care product of claim 1, characterized in that the licorice flavonoid extract is in an amount of 0.05 wt %-0.5 wt %.

6. An oral pharmaceutical or oral care product, characterized in comprising Licochalone A without comprising other licorice extracts.

7. The oral pharmaceutical or oral care product of claim 6, characterized in that the Licochalone A is in an amount of 0.0005 wt %-20 wt %.

8. The oral pharmaceutical or oral care product of claim 6, characterized in that the Licochalone A is in an amount of 0.001 wt %-10 wt %.

9. The oral pharmaceutical or oral care product of claim 6, characterized in that the Licochalone A is in an amount of 0.025 wt %-5 wt %.

10. The oral pharmaceutical or oral care product of claim 6, characterized in that the Licochalone A is in an amount of 0.05 wt %-0.5 wt %.

11. The oral pharmaceutical or oral care product of claim 1, characterized in that the oral pharmaceutical or oral care product is tooth paste, mouthwash, paste, rapidly disintegrating tablet, pellicle or chewing gum.

12. Use of Licochalone A in manufacturing oral pharmaceutical or oral care products for inhibiting primary oral pathogenic microorganisms, preventing caries, countering breath odor, reducing dental plaques, reducing gingivitis, tooth desensitizing, anti-calculus or tooth whitening.

13. Use of licorice flavonoid extract in manufacturing oral pharmaceuticals or oral care products or inhibiting primary oral pathogenic microorganisms, preventing dental caries, countering breath odor, reducing dental plaques, reducing gingivitis, tooth desensitizing, anti-calculus, or tooth whitening, wherein the licorice is selected from Glycyrrhiza inflate Betal, Glycyrrhiza glabra L. and Glycyrrhiza kansuensis, and a mixture thereof.
